(19) United States
(12) Patent Application Publication
(10) Pub. No.: US 2025/0213475 A1
(43) Pub. Date: Jul. 3, 2025

(54) LIPOSOMAL COMPOSITIONS

(71) Applicant: R.G.C.C. Holdings AG, Zug (CH)

(72) Inventor: Ioannis PAPASOTIRIOU, Oberägeri (CH)

(73) Assignee: R.G.C.C. Holdings AG, Zug (CH)

(21) Appl. No.: 18/848,181

(22) PCT Filed: Mar. 16, 2023
(86) PCT No.: PCT/EP2023/056805

§ 371 Date: Sep. 18, 2024

(30) Foreign Application Priority Data
Mar. 21, 2022 (EP) 22163361.3

(51) Int. Cl. A61K 9/1271; A61K 31/517; A61K 31/52; A61K 31/675; A61P 35/00

(52) U.S. Cl. CPC A61K 9/1271; A61K 31/517; A61K 31/52; A61K 31/675; A61P 35/00

(57) ABSTRACT

A pharmaceutical composition including a nucleoside analogue loaded into liposomes of a liposomal delivery system, wherein the nucleoside analogue is a purine analogue chosen from {[2-(6-amino-9H-purin-9-yl)quinazolin-4-yl]oxy}phosphonic acid or 2-(6-amino-9H-purin-9-yl) quinazolin-4-ol, or a pyrimidine analogue chosen from 4-amino-1-(4-hydroxyquinazolin-2-yl)-1,2-dihydropyrimidin-2-one or N-[1-(4-hydroxyquinazolin-2-yl)-2-oxo-1,2-dihydropyrimidin-4-yl]acetamide.

### TECHNICAL FIELD

The present invention relates to liposomal compositions, in particular liposomal compositions incorporating nucleoside analogues, and their use in cancer treatment.

### PRIOR ART

Cancer, one of the leading causes of death worldwide, continues to be the focus of many research efforts.

While new potentially therapeutic molecules as well as new targets are discovered, the key to effectively treat a certain disorder, such as cancer, often depends on enhancing the delivery of an active ingredient to the targeted cells.

Innovative drug delivery systems that are capable of such delivery are needed and under rigorous research to provide biocompatible, cost effective, safe and efficient options that allow their use in therapy.

A prominent drug delivery system is that of liposomes, which can be loaded in their interior with active ingredients. Liposomes are nano-sized, and can be modified on their surface in order to be more targeted or multiple types of liposomes can be combined for improved results.

Liposomes are a well-established tool for drug delivery and were first described by Alee Bangham in 1963.

Today, there exist already products in liposomal form available commercially like Onivyde™, Marqibo®, Doxil®, Visudyne® and Depocyt®, which are useful in cancer therapy.

Liposomes encapsulate both hydrophilic and hydrophobic drugs. They are mainly composed of phospholipids, which can form single layers or bilayers in most environments. Hydrophilic drugs are usually entrapped in the interior of liposomes (aqueous environment) whereas hydrophobic molecules stay within the lipid bilayers of liposomes. When phospholipids disperse at an aqueous environment at temperatures above their transition temperature they tend to form particles with an aqueous core.

Liposomes have an additional advantage when used in combination with cytotoxic agents. The compartmentalization provided by the liposomes can reduce toxicity while at the same time offering a more targeted drug delivery and thus, increased efficacy.

WO2017155923A1 relates to nucleotide or nucleoside therapeutic compositions as well as their use in treating infectious diseases, viral infections, and cancer, and discloses that liposomal suspensions may be appropriate for the delivery of free nucleosides, acyl nucleosides or phosphate ester prodrug forms of the nucleoside compounds according to the present invention.

WO2015038596A1 relates to further to sulfur-containing nucleotide or nucleoside therapeutic compositions as well as their use in treating infectious diseases, viral infections, and cancer, and discloses that liposomal suspensions may be appropriate for the delivery of free nucleosides, acyl nucleosides or phosphate ester prodrug forms of the nucleoside compounds according to the present invention.

WO2010014895A2 relates to nucleic acids, such as oligonucleotides, that are encapsulated within liposomes complexes containing a mixture of a cationic lipid, a fusogenic lipid and a PEG lipid.

WO2006029081A2 relates to nucleoside-lipid conjugates comprising nucleosides and nucleoside analogues such as gemcitabine conjugated with phospholipids and further relates to the use of such nucleoside-lipid conjugates for the treatment of mammalian diseases, such as cancer and viral infections. The nucleoside-lipid conjugates may be used in the preparation of liposomes together with phospholipids such as phosphatidylcholine, phosphatidylethanolamine, phosphatidylglycerol, phosphatidic acid, cholesterol, [alpha]-tocopherol, natural or synthetic cardiolipin and analogues thereof, synthetic cationic cardiolipin analogues and derivatives thereof, phosphatidylserine, phosphatidylinositol, sphingomyelin, lysophosphotidylglycerol, lysophosphatidic acid, lysophosphotidylcholine, lysophosphatidylserine and PEG modified lipids.

WO2011062503A1 relates to parenteral formulations for certain long chain saturated and monounsaturated fatty acid derivatives of 2′,2′-difluorodeoxycytidine (Gemcitabine), a nucleoside analog, in the treatment of cancer, and further relates to a parenteral pharmaceutical composition said derivatives in the form of liposomes having an average particle size in the range of 2.5-30 nm.

US20030026831 A1 relates to a pharmaceutical composition comprising: (a) an anionic liposome comprising a phospholipid with a head group selected from the group consisting of sn-glycero-phosphocholine; sn-glycero-phospho-rac-(1-glycer-ol); sn-glycero-phospho-L-serine; sn-glycero-3-phosphate; and combinations thereof; (b) a bioactive agent, which may be a nucleoside analogue; and (c) a cation, a buffer, or a combination thereof; wherein the anionic liposome is not a combination of 1,2-dioleoyl-sn-glycero-3-phosphocholine (DOPC) and 1,2-dioleoyl-sn-glycero-3-[phospho-L-serine] (DOPS) in a ratio of 2:1 having a diameter of 122 nm to 162 nm.

Saraf, Shivani, et al. “Advances in liposomal drug delivery to cancer: An overview.” Journal of drug delivery science and technology 56 (2020): 101549 is a review publication that discloses liposomes in cancer therapy, for example for liposomes of paclitaxel, triptolide, resveratrol, doxorubicin, docetaxel, hydroxyquinone, curcumin, sorafenib, shikonin, losartan, and gemcitabine, 5-fluorouracil, cordycepin. Gemcitabine is a pyrimidine analogue, in particular a nucleoside analogue, in which the hydrogen atoms on the 2′ carbon of deoxycytidine are replaced by fluorine atoms. 5-fluorouracil is a pyrimidine analogue, in particular a nucleobase analogue. Cordycepin is a purine analogue, in particular a is a adenosine analogue.

### SUMMARY OF THE INVENTION

It is therefore an object of the present invention to provide a pharmaceutical composition comprising a nucleoside analogue loaded into liposomes of a liposomal delivery system, wherein the nucleoside analogue is a purine analogue chosen from {[2-(6-amino-9H-purin-9-yl)quinazolin-4-yl]oxy}phosphonic acid or 2-(6-amino-9H-purin-9-yl)quinazolin-4-ol, or a pyrimidine analogue chosen from 4-amino-1-(4-hydroxyquinazolin-2-yl)-1,2-dihydropyrimidin-2-one or N-[1-(4-hydroxyquinazolin-2-yl)-2-oxo-1,2-dihydropyrimidin-4-yl]acetamide. The aforementioned nucleoside analogues were hitherto unknown for use in a pharmaceutical composition, in particular for use in the treatment of cancer.

The chemical structure of the nucleoside analogues are depicted below.

| Name | Chemical structure |
|---|---|
| {[2-(6-amino-9H-purin-9- yl)quinazolin-4-yl]oxy}phosphonic acid | 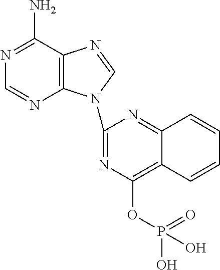 [SMILES: Nc1ncnc2c1ncn2-c1nc(OP(=O)(O)O)c2ccccc2n1] |
| 4-amino-1-(4-hydroxyquinazolin- 2-yl)-1,2-dihydropyrimidin-2-one | 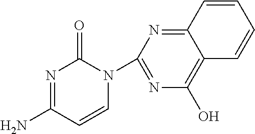 [SMILES: Nc1ccn(-c2nc(O)c3ccccc3n2)c(=O)n1] |
| 2-(6-amino-9H-purin-9- yl)quinazolin-4-ol | 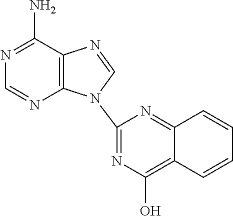 [SMILES: Nc1ncnc2c1ncn2-c1nc(O)c2ccccc2n1] |
| N-[1-(4-hydroxyquinazolin-2-yl)-2- oxo-1,2-dihydropyrimidin-4- yl]acetamide | 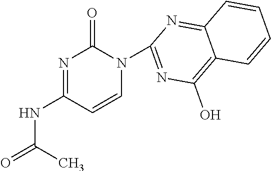 [SMILES: CC(=O)Nc1ccn(-c2nc(O)c3ccccc3n2)c(=O)n1] |

In an embodiment of the pharmaceutical composition according to the above object of the invention, the nucleoside analogue is covalently coupled or is not covalently coupled to the liposome.

In an embodiment of the pharmaceutical composition according to the above object of the invention, the liposomes of the liposomal delivery system have a diameter of at least 750, preferably of at least 775, and more preferably of at least 800 nm, and preferably of from 800 nm to 1000 nm, or 800 to 950 nm, as determined by SEM. Liposomes having the preferred size are more stable than liposomes having smaller sizes, especially than smaller liposomes having a size in the range 10 to 100 nm.

In an embodiment of the pharmaceutical composition according to the above object of the invention, the liposomes of the liposomal delivery system are sterically stabilized liposomes.

In an embodiment of the pharmaceutical composition according to the above object of the invention, the liposomes of the liposomal delivery system comprise at least a phospholipid, preferably at least a phosphatidylcholine and/or a phosphatidylethanolamine, or comprise at least a phospholipid, more preferably at least a phosphatidylcholine and/or a phosphatidylethanolamine, most preferably in addition to one or more phosphoglycerides and/or one or more sphingolipids. The inclusion of phosphatidylethanolamine and phosphatidylcholine has the advantage of providing amino or trimethylamino groups on the surfaces of the liposomes, which groups may be useful for further functionalizing the liposomes, for example via the formation of amide bonds in case of proteins or amino acids.

In an embodiment of the pharmaceutical composition according to the above object of the invention, the liposomes of the liposomal delivery system comprise, or essentially consist of, as the lipid component(s) of the liposomes, a pegylated phospholipid such as pegylated 1,2-distearoyl-sn-glycero-3-phosphoethanolamine, or a combination of a pegylated and an unpegylated phospholipid such as such as pegylated 1,2-distearoyl-sn-glycero-3-phosphoethanolamine and unpegylated phosphatidylcholine, respectively, optionally together with a sterol such as cholesterol. It has been found that liposomes of the aforementioned composition have better incorporation efficiency during the production process.

In an embodiment of the pharmaceutical composition according to the above object of the invention, the pegylated phospholipid is pegylated phosphoethanolamine and the unpegylated phospholipid is a phosphatidylcholine. It has been found that liposomes of the aforementioned composition have better incorporation efficiency during the production process.

In an embodiment of the pharmaceutical composition according to the above object of the invention, the liposomes of the liposomal delivery system are free of fatty acids in particular free of unsaturated, and preferably of monounsaturated, fatty acids such as oleic acid. It has been found that when liposomes are free of fatty acids, incorporation efficiency during the production process is enhanced.

In an embodiment of the pharmaceutical composition according to the above object of the invention, the nucleoside is {[2-(6-amino-9H-purin-9-yl)quinazolin-4-yl]oxy}phosphonic acid, and the liposomes of the liposomal delivery system comprise, or consists of, a pegylated phospholipid as the sole phospholipid component of the liposome, such as for example N-(carbonyl-methoxypolyethylene glycol-2000)-1,2-distearoyl-sn-glycero-3 phosphoethanolamine and a sterol, such as cholesterol. In that embodiment, in the liposome, the amount of pegylated phospholipid may of from about 85 wt % to about 95 wt %, more preferably of from about 90 wt % to about 95 wt %, based on the total weight of the pegylated phospholipid and the sterol. In that embodiment, in the liposome, the amount of sterol may of from about 5 wt % to about 15 wt %, more preferably of from about 5 wt % to about 10 wt %, based on the total weight of the pegylated phospholipid and the sterol.

In an embodiment of the pharmaceutical composition according to the above object of the invention, the liposomes of the liposomal delivery system comprise an amount of sterol such as cholesterol of from about more than 5 wt % to less than 50 wt %, preferably of from about more than 5 wt % to less than 25 wt %, more preferably of from about more than 5 wt % to less than 20 wt %, most preferably of from about more than 6 wt % to less than 16 wt %, based on the weight of the pegylated phospholipid or the weight of the combination of a pegylated and an unpegylated phospholipid.

In an embodiment of the pharmaceutical composition according to the above object of the invention, the liposomes of the liposomal delivery system comprises, or consists of, of from 40 to 45 wt % of a pegylated phospholipid such as N-(carbonyl-methoxypolyethylene glycol-2000)-1,2-distearoyl-sn-glycero-3 phosphoethanolamine, of from 40 to 45 wt % of an unpegylated phospholipid such as phosphatidylcholine, and of a sterol such as cholesterol, based on the total weight of the pegylated phospholipid, unpegylated phospholipid and sterol, and preferably wherein the pegylated phospholipid and unpegylated phospholipid are comprised at a weight ratio of 1:1.

It is a further object of the present invention to provide a pharmaceutical composition according to the above object for use in the treatment of colon cancer or lung cancer or breast cancer, preferably via oral or parenteral route.

It is yet a further object of the present invention to provide a Use of a pharmaceutical composition according to the above object for the inhibition of proliferation of cancer cell lines in vitro, preferably in a concentration of 0.01 μM to 10 μM and/or wherein the cancer cell line is preferably a lung or colon or breast cancer cell line and/or the nucleoside analog is {[2-(6-amino-9H-purin-9-yl)quinazolin-4-yl]oxy}phosphonic acid or 4-amino-1-(4-hydroxyquinazolin-2-yl)-1,2-dihydropyrimidin-2-one, and is preferably present in a concentration of 0.01 μM to 1 μM.

It is another object of the present invention to provide a process for producing a liposomal delivery system comprising a nucleoside analogue loaded into liposomes, wherein the liposomes of the liposomal delivery system comprise, or consist of, a combination of a pegylated and an unpegylated phospholipid, and preferably of from 40 to 45 wt % of a pegylated phospholipid such as N-(carbonyl-methoxypolyethylene glycol-2000)-1,2-distearoyl-sn-glycero-3 phosphoethanolamine, of from 40 to 45 wt % of an unpegylated phospholipid such as phosphatidylcholine, and of a sterol such as cholesterol, based on the total weight of the pegylated phospholipid, unpegylated phospholipid and sterol, and more preferably wherein the pegylated phospholipid and unpegylated phospholipid are comprised at a weight ratio of 1:1, or 4:5 to 5:4, and wherein the nucleoside analogue is chosen among 4-amino-1-(4-hydroxyquinazolin-2-yl)-1,2-dihydropyrimidin-2-one, or N-[1-(4-hydroxyquinazolin-2-yl)-2-oxo-1,2-dihydropyrimidin-4-yl]acetamide, wherein the process comprises the steps of:
a. solubilizing the pegylated phospholipid, unpegylated phospholipid, and sterol, and the nucleoside analogue, in an polar solvent, preferably in a mixture of polar solvents,
b. evaporating the solvent, and
c. adding an aqueous solution or water, preferably an aqueous buffer solution, such as to form a first liposomal suspension, and
d. sonicating the first liposomal suspension to form a second liposomal suspension,
e. annealing the second liposomal suspension to form the liposomal delivery system comprising a nucleoside analogue loaded into liposomes.

It is another object of the present invention to provide a process for producing a liposomal delivery system comprising a nucleoside analogue loaded into liposomes, wherein the liposomes of the liposomal delivery system comprise, or consist of, a combination of a pegylated and an unpegylated phospholipid, and preferably of from 40 to 45 wt % of a pegylated phospholipid such as N-(carbonyl-methoxypolyethylene glycol-2000)-1,2-distearoyl-sn-glycero-3 phosphoethanolamine, of from 40 to 45 wt % of an unpegylated phospholipid such as phosphatidylcholine, and of a sterol such as cholesterol, based on the total weight of the pegylated phospholipid, unpegylated phospholipid and sterol, and more preferably wherein the pegylated phospholipid and unpegylated phospholipid are comprised at a weight ratio of 1:1, or 4:5 to 5:4, and wherein the nucleoside analogue is chosen among {[2-(6-amino-9H-purin-9-yl)quinazolin-4-yl]oxy}phosphonic acid and 2-(6-Amino-9H-purin-9-yl)quinazolin-4-ol, wherein the process comprises the steps of:
a. solubilizing the pegylated phospholipid, unpegylated phospholipid, and sterol, in an polar solvent, preferably in a mixture of polar solvents,
b. evaporating the solvent, and
c. adding an aqueous solution of the nucleoside analogue, preferably an aqueous buffer solution of the nucleoside analogue, to form a first liposomal suspension, and
d. sonicating the first liposomal suspension to form a second liposomal suspension,
e. annealing the second liposomal suspension to form the liposomal delivery system comprising a nucleoside analogue loaded into liposomes.

It is another object of the present invention to provide a process for producing a liposomal delivery system comprising a nucleoside analogue loaded into liposomes,
wherein the liposomes of the liposomal delivery system comprise, or consist of, a pegylated phospholipid as the sole phospholipid component of the liposome, such as N-(carbonyl-methoxypolyethylene glycol-2000)-1,2-distearoyl-sn-glycero-3 phosphoethanolamine, and of a sterol such as cholesterol, where in the liposome, preferably the amount of pegylated phospholipid is of from about 85 wt % to about 95 wt %, more preferably of from about 90 wt % to about 95 wt %, based on the total weight of the pegylated phospholipid and the sterol, and/or in the liposome, the amount of sterol is of from about 5 wt % to about 15 wt %, more preferably of from about 5 wt % to about 10 wt %, based on the total weight of the pegylated phospholipid and the sterol, and
wherein the nucleoside analogue is {[2-(6-amino-9H-purin-9-yl)quinazolin-4-yl]oxy}phosphonic acid,
wherein the process comprises the steps of:
a. solubilizing the pegylated phospholipid and sterol, in an polar solvent, preferably in a mixture of polar solvents,
b. evaporating the solvent, and
c. adding an aqueous solution of the nucleoside analogue, preferably an aqueous buffer solution of the nucleoside analogue, such as to form a first liposomal suspension, and
d. sonicating the first liposomal suspension to form a second liposomal suspension,
e. annealing the second liposomal suspension to form the liposomal delivery system comprising a nucleoside analogue loaded into liposomes.

Further embodiments of the invention are laid down in the dependent claims.

### BRIEF DESCRIPTION OF THE DRAWINGS

Preferred embodiments of the invention are described in the following with reference to the drawings, which are for the purpose of illustrating the present preferred embodiments of the invention and not for the purpose of limiting the same. In the drawings,

FIG. 1 shows a flow chart that depicts an embodiment of the claimed process, in which the encapsulation of 4-amino-1-(4-hydroxyquinazolin-2-yl)-1,2-dihydropyrimidin-2-one, and N-[1-(4-hydroxyquinazolin-2-yl)-2-oxo-1,2-dihydropyrimidin-4-yl]acetamide into the liposomal delivery is shown. Note that for 4-Amino-1-(4-hydroxyquinazolin-2-yl)-1,2-dihydropyrimidin-2-one, and N-[1-(4-hydroxyquinazolin-2-yl)-2-oxo-1,2-dihydropyrimidin-4-yl]acetamide, the nucleoside analogue is added at the stage of dissolution of the lipid components of the liposomal delivery system in chloroform:methanol.

FIG. 2 shows a flow chart that depicts an embodiment of the claimed process, in which the encapsulation of {[2-(6-amino-9H-purin-9-yl)quinazolin-4-yl]oxy}phosphonic acid and 2-(6-Amino-9H-purin-9-yl)quinazolin-4-ol into the liposomal delivery is shown. Note that for {[2-(6-amino-9H-purin-9-yl)quinazolin-4-yl]oxy}phosphonic acid and 2-(6-Amino-9H-purin-9-yl)quinazolin-4-ol, the nucleoside analogue is added at the stage of hydration of the thin film with PBS, the nucleoside analogue being dissolved in PBS.

FIG. 3 shows standard curves for of the different nucleoside analogues in phosphate buffer saline (PBS) for {[2-(6-amino-9H-purin-9-yl)quinazolin-4-yl]oxy}phosphonic acid, for 2-(6-Amino-9H-purin-9-yl)quinazolin-4-ol, for N-[1-(4-hydroxyquinazolin-2-yl)-2-oxo-1,2-dihydropyrimidin-4-yl]acetamide, for 4-amino-1-(4-hydroxyquinazolin-2-yl)-1,2-dihydropyrimidin-2-one

FIG. 4 shows a differential scanning calorimetry thermographs of the phase transition peaks of liposomes of (1): 4-amino-1-(4-hydroxyquinazolin-2-yl)-1,2-dihydropyrimidin-2-one in liposomes of the liposomal delivery system 3, (2): blank liposomes of the liposomal delivery system 3, (3): 2-(6-Amino-9H-purin-9-yl)quinazolin-4-ol in liposomes of the liposomal delivery system 3, (4): blank liposomes of the liposomal delivery system 2, (5): N-[1-(4-hydroxyquinazolin-2-yl)-2-oxo-1,2-dihydropyrimidin-4-yl]acetamide in liposomes of the liposomal delivery system 3, (6): {[2-(6-amino-9H-purin-9-yl)quinazolin-4-yl]oxy}phosphonic acid in liposomes of the liposomal delivery system 2.

FIG. 5 shows SEM images of blank liposomes, with 1a and 1b are pictures of liposome blank1 with diameters of 642, 630, and 668 nm as can be seen in 1a, which is an enlarged portion of 1b and 2a and 2b are pictures of liposome blank 2 with diameters of 642, 630, and 668 nm.

FIG. 6 shows SEM images of (A): liposomes encapsulating (A): 2-(6-Amino-9H-purin-9-yl)quinazolin-4-yl dihydrogen phosphate, with an enlarged section of the SEM image of (A) shown in (B). In (B), it can be seen that the diameter of three selected liposomes is determined to be 807, 816 and 838 nm.

FIG. 7 shows SEM images of (A): liposomes encapsulating (A): 2-(6-Amino-9H-purin-9-yl)quinazolin-4-ol, with an enlarged section of the SEM image of (A) shown in (B). In (B), it can be seen that the diameter of three selected liposomes is determined to be 938, 907 and 925 nm.

FIG. 8 shows SEM images of (A): liposomes encapsulating (A); and N-(9-(4-Hydroxyquinazolin-2-yl)-6-oxo-6,9-dihydro-1H-purin-2-yl) acetamide, with an enlarged section of the SEM image of (A) shown in (B). In (B), it can be seen that the diameter of three selected liposomes is determined to be 860, 830 and 755 nm.

FIG. 9 shows SEM images of (A): liposomes encapsulating 4-amino-1-(4-hydroxyquinazolin-2-yl)-1,2-dihydropyrimidin-2-one, with an enlarged section of the SEM image of (A) shown in (B). In (B), it can be seen that the diameter of three selected liposomes is determined to be 862, 846 and 834 nm.

### DESCRIPTION OF PREFERRED EMBODIMENTS

In an embodiment of the pharmaceutical composition according to the above object of the invention, the nucleoside analogue is covalently coupled or is not covalently coupled to the liposome.

In an embodiment of the pharmaceutical composition according to the above object of the invention, the liposomes of the liposomal delivery system have a diameter of at least 750, preferably of at least 775, and more preferably of at least 800 nm, and preferably of from 800 nm to 1000 nm, or 800 to 950 nm, as determined by SEM. Liposomes having the preferred size are more stable than liposomes having smaller sizes, especially than smaller liposomes having a size in the range 10 to 100 nm.

In an embodiment of the pharmaceutical composition according to the above object of the invention, the liposomes of the liposomal delivery system are sterically stabilized liposomes.

In an embodiment of the pharmaceutical composition according to the above object of the invention, the liposomes of the liposomal delivery system comprise at least a phospholipid, preferably at least a phosphatidylcholine and/or a phosphatidylethanolamine, or comprise at least a phospholipid, more preferably at least a phosphatidylcholine and/or a phosphatidylethanolamine, most preferably in addition to one or more phosphoglycerides and/or one or more sphingolipids. The inclusion of phosphatidylethanolamine and phosphatidylcholine has the advantage of providing amino or trimethylamino groups on the surfaces of the liposomes, which groups may be useful for further functionalizing the liposomes, for example via the formation of amide bonds in case of proteins or amino acids.

In an embodiment of the pharmaceutical composition according to the above object of the invention, the liposomes of the liposomal delivery system comprise, or essentially consist of, as the lipid component(s) of the liposomes, a pegylated phospholipid such as pegylated 1,2-distearoyl-sn-glycero-3-phosphoethanolamine, or a combination of a pegylated and an unpegylated phospholipid as such pegylated 1,2-distearoyl-sn-glycero-3-phosphoethanolamine and unpegylated phosphatidylcholine, respectively, optionally together with a sterol such as cholesterol. It has been found that liposomes of the aforementioned composition have better incorporation efficiency during the production process.

In an embodiment of the pharmaceutical composition according to the above object of the invention, the pegylated phospholipid is pegylated phosphoethanolamine and the unpegylated phospholipid is a phosphatidylcholine. It has been found that liposomes of the aforementioned composition have better incorporation efficiency during the production process.

In an embodiment of the pharmaceutical composition according to the above object of the invention, the liposomes of the liposomal delivery system are free of fatty acids in particular free of unsaturated, and preferably of monounsaturated, fatty acids such as oleic acid. It has been found that when liposomes are free of fatty acids, incorporation efficiency during the production process is enhanced.

In an embodiment of the pharmaceutical composition according to the above object of the invention, the nucleoside is {[2-(6-amino-9H-purin-9-yl)quinazolin-4-yl]oxy}phosphonic acid, and the liposomes of the liposomal delivery system comprise, or consists of, a pegylated phospholipid as the sole phospholipid component of the liposome, such as for example N-(carbonyl-methoxypolyethylene glycol-2000)-1,2-distearoyl-sn-glycero-3 phosphoethanolamine and a sterol, such as cholesterol. In that embodiment, in the liposome, the amount of pegylated phospholipid may of from about 85 wt % to about 95 wt %, more preferably of from about 90 wt % to about 95 wt %, based on the total weight of the pegylated phospholipid and the sterol. In that embodiment, in the liposome, the amount of sterol may of from about 5 wt % to about 15 wt %, more preferably of from about 5 wt % to about 10 wt %, based on the total weight of the pegylated phospholipid and the sterol.

In an embodiment of the pharmaceutical composition according to the above object of the invention, the liposomes of the liposomal delivery system comprise an amount of sterol such as cholesterol of from about more than 5 wt % to less than 50 wt %, preferably of from about more than 5 wt % to less than 25 wt %, more preferably of from about more than 5 wt % to less than 20 wt %, most preferably of from about more than 6 wt % to less than 16 wt %, based on the weight of the pegylated phospholipid or the weight of the combination of a pegylated and an unpegylated phospholipid.

In an embodiment of the pharmaceutical composition according to the above object of the invention, the liposomes of the liposomal delivery system comprises, or consists of, of from 40 to 45 wt % of a pegylated phospholipid such as N-(carbonyl-methoxypolyethylene glycol-2000)-1,2-distearoyl-sn-glycero-3 phosphoethanolamine, of from 40 to 45 wt % of an unpegylated phospholipid such as phosphatidylcholine, and of a sterol such as cholesterol, based on the total weight of the pegylated phospholipid, unpegylated phospholipid and sterol, and preferably wherein the pegylated phospholipid and unpegylated phospholipid are comprised at a weight ratio of 1:1.
use in a pharmaceutical composition, in particular for use in the treatment of cancer.

It is understood that the above limitations to the composition apply also to the process for producing a liposomal delivery system comprising any of the nucleoside analogues.

In a preferred embodiment of the process for producing a liposomal delivery system comprising any of the nucleoside analogues loaded into liposomes, the polar solvent comprises chloroform, preferably mixed with ethanol or methanol, in a ratio of 3:1.

In a preferred embodiment of the process for producing a liposomal delivery system comprising any of the nucleoside analogues loaded into liposomes, annealing of the second liposomal suspension to form the liposomal delivery system comprising a nucleoside analogue loaded into liposomes is carried out at a temperature of at least 45° C., more preferably at a temperature of more than 50° C. and most preferably at a temperature of between 55 and 75° such as for example at 65° C.

In a preferred embodiment of the process for producing a liposomal delivery system comprising any of the nucleoside analogues loaded into liposomes, the aqueous buffer solution is phosphate saline buffer, and preferably is a phosphate saline buffer having a neutral pH.

In a preferred embodiment of the process for producing a liposomal delivery system comprising any of the nucleoside analogues loaded into liposomes, the concentration of the nucleoside analogues in step c. is about 1 mg/mL in the first liposomal suspension.

### EXAMPLES

### Materials and Methods

LIPOID PE 18:0/18:0-PEG 2000, hereinafter DSPE-PEG, ([N-(carbonyl-methoxypolyethylene glycol-2000)-1,2-distearoyl-sn-glycero-3 phosphoethanolamine, Sodium Salt]) and LIPOID S PC-3, hereinafter PC, (Hydrogenated phosphatidylcholine from soybean) were purchased by Lipoid (Lipoid GmbH, Frigenstraße 4, D-67065 Ludwigshafen, Germany). Cholesterol, hereinafter CHOL, and Oleic acid, hereinafter OA, were purchased by Fluorochem (Graphite Way, Hadfield, Derbyshire, UK). Phosphate Buffer Saline 0.01M and pH 7.4 was purchased from Sigma-Aldrich (3050 Spruce Street, Saint Louis, MO 63103, USA) as well as Methanol and Chloroform.

### Preparation of Liposomes

Liposomes were prepared based on the thin film method. Different lipid concentrations were tested and based on their encapsulation efficacy, the ideal system was selected for each occasion. 2-(6-Amino-9H-purin-9-yl)quinazolin-4-ol and {[2-(6-amino-9H-purin-9-yl)quinazolin-4-yl]oxy}phosphonic acid were initially encapsulated into different systems and the ones that exhibited best results were used for the rest APIs. All the concentrations that were used, can be seen at Table 1.

TABLE 1
Description of the different recipes that were used during the
development of encapsulated nucleoside analogues into liposomes

| | | PC | DSPE-PEG | Cholesterol | Oleic |
|---|---|---|---|---|---|
| | | (100 mg/mL), | (100 mg/mL), | (20 mg/mL), | Acid, |
| Code | Composition | μL | μL | μL | μL |
| 1 | DSPE- | — | 250 | 625 | 18.76 |
| | PEG:CHOL:OA | | | | |
| 2 | DSPE-PEG:CHOL | — | 250 | 85 | — |
| 3 | DSPE- | 125 | 125 | 196 | |
| | PEG:PC:CHOL | | | | |
| 4 | DSPE- | 125 | 125 | 625 | 18.76 |
| | PEG:CHOL:OA:PC | | | | |
| 5 | DSPE-PEG:OA | — | 250 | — | 18.76 |

Briefly, the preparation of liposomes began with the dissolution of PE 18:0/18:0-PEG 2000, Phosphatidylcholine (PC) and Cholesterol into 10 mL of a solution of chloroform:methanol (3:1) in a 50 mL round bottom flask. Then, the solvent was removed through vacuum evaporation, until it was completely dried or in some cases for the complete evaporation of the solvent, nitrogen stream was used. After that, a thin film of lipid is formed at the walls of the flask, which is hydrated with 10 mL Phosphate Buffer Saline (PBS) 0.01M pH 7.4, sonicated for 30 minutes at a sonication bath and then left to anneal its lipids at 65° C.

### Nucleoside Analogues Encapsulation

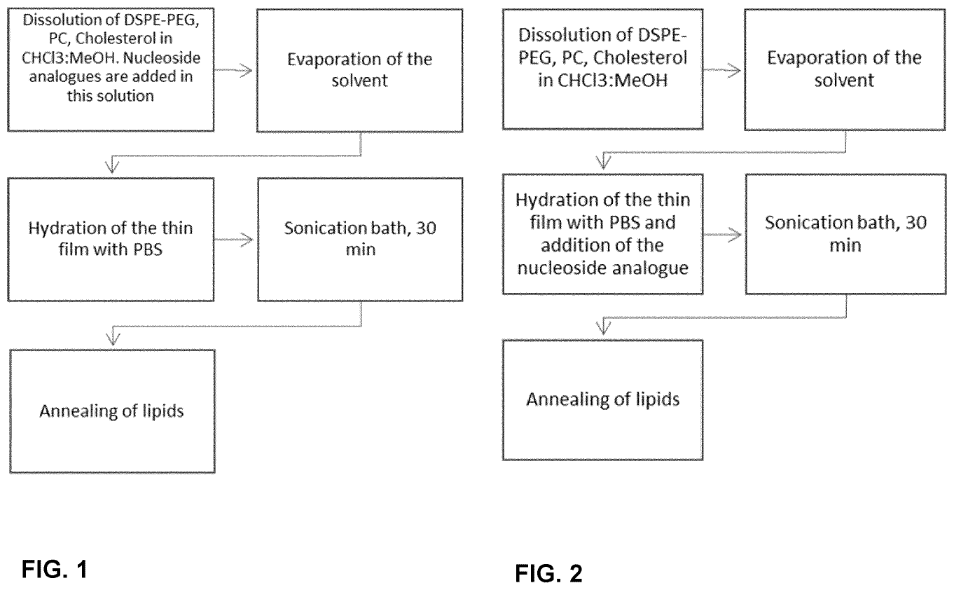

Nucleoside analogues were encapsulated based on their nature at different steps of the process. 4-amino-1-(4-hydroxyquinazolin-2-yl)-1,2-dihydropyrimidin-2-one, and N-[1-(4-hydroxyquinazolin-2-yl)-2-oxo-1,2-dihydropyrimidin-4-yl]acetamide were added initially at the process, along with the lipids and chloroform:methanol solution. To improve the solubility of the analogues, magnetic stirring was applied prior to vacuum evaporation (Process 1). On the other hand, {[2-(6-amino-9H-purin-9-yl)quinazolin-4-yl]oxy}phosphonic acid and 2-(6-Amino-9H-purin-9-yl)quinazolin-4-ol were added after their dissolution in PBS solution after the evaporation of the organic solvent (Process 2). The concentration of the nucleoside analogues was in all cases 1 mg/mL. FIGS. 1 and 2 depict a schematic of liposome preparation according to Process 1 and 2, respectively.

As comparative examples, the same process as above was followed, but without adding any nucleoside analogues.

### Liposomes Purification

At the end of the above encapsulation process, the solution was centrifuged at 15.000 rpm for 20 minutes and the supernatant was kept for indirect encapsulation determination. The precipitate was lyophilized and kept for further analysis and characterization.

### Freeze Drying

Glass vials with rubber stoppers were inserted in the Freeze Dryer (Telstar, LyoBeta Mini) with liposomes precipitate in PBS. The freeze-drying process started with freezing for 2.5 h at −40° C. and hold of this temperature for other 3 h. Then the condenser preparation starts and lasts for 10 minutes. After that, the vacuum in the chamber begins, at 0.5 mbar, in order to start drying. Primary drying lasts 2 h and 13 min at 0° C. and 0.5 mbar. These conditions are hold for 11h (primary drying hold) and then secondary drying starts. Temperature during secondary drying is 23° C., 0.1 mbar and lasts 6 h and 17 min. Finally, cycle ends.

### Encapsulation Efficacy

The supernatants after each centrifugation were kept and used to evaluate the amount of the free drug. Subtracting the amount of the free drug at the supernatant from the total added drug, the rest is considered as encapsulated. Briefly, from each solution samples were collected in order to prepare solutions, which were then evaluated with UV/VIS spectrometry. Based on standard curves, in which the different nucleosides were measured in solution on a UV-VIS spectrometer, different the remaining quantity was calculated. The encapsulation efficacy was measured then based on the following equation:

$$\frac{Quantity of the Nucleoside Analogue in the liposome}{Quantity of the initially added nucleoside analogue} \times 100 \qquad ( 1 )$$

Quantity of the Nucleoside Analogue in the liposome=(Quantity of the Nucleoside Analogue initially added−Quantity of the Nucleoside Analogue in the supernatant).

### Yield

After every completion of the freeze drying protocol, the dry powder of each product was weighed. The yield of each one was calculated based on the following equation (2):

$$\frac{Mass of the powder after freeze drying}{Mass of the intially added compounds} \times 100 \% \qquad ( 2 )$$

### Scanning Electron Microscopy

Liposomes size, shape and appearance were evaluated using Scanning Electron Microscopy (SEM), Phenom ProX (Thermo Fischer Scientific). PELCO Tabs™ Carbon Conductive Tabs, Double coated, 12 mm OD were used with aluminum specimen mounts (½″ slotted head and ⅛″ pin). These tabs are adhesives that are used in SEM mounting for better conductivity. The chosen mode is mentioned as “all materials (5 kV)”, with an image intensity and BSD full detector. Live viewing has a resolution of 1024 and medium quality where the acquired image has 2048 resolution and high quality. The software version is 4.5.2. All image processing (photo acquisition, size analysis etc.) was done through the instrument's software.

### Differential Scanning Calorimetry

In general, thermal analysis measures the changes in properties of a material as a function of temperature. In this study, Differential Scanning calorimetry was used, which measures the heat absorbed or liberated during heating or cooling. This happens through measuring the difference in heat flow rate between the sample and an inert reference under controlled atmosphere in correlation with time and temperature. DSC is a powerful tool when it comes to determining physical and chemical changes of materials and systems as a result of endothermic or exothermic events. The processes can be both exothermic or endothermic, depending whether the sampling is releasing or absorbing energy respectively. The basic principle of the method is that at physical transformations such as phase transition, more or less heat will need to flow to the sample than to the reference material to maintain both at the same temperature.

Differential Scanning calorimetry (DSC) is a useful tool when it comes to liposome characterization. These colloidal lyotropic systems can show their properties through different temperature changes. The program used for this study starts with heating of the sample from 30° C. to 180° C. with a rate of 10° C./min and hold there for 1 minute. Then, heat is applied again from 180° to 370° C. The experiment is performed using DSC3, Mettler Toledo. The inert gas is N₂, with a rate of 50 mL/min. Previously, the instrument was calibrated with a standard method using Indium as standard material. The software, with which the results are evaluated, is STARe Software.

### Cytotoxicity Assay

The human breast adenocarcinoma (luminal type) MCF7 cell line (ECACC 86012803), human breast adenocarcinoma (triple negative) MDA-MB-231 cell line (ECACC 92020424), human colorectal carcinoma HCT116 cell line (ECACC 91091005) and human lung cancer COLO699N cell line (ECACC 93052608) were culture in RPMI 1640 medium Catalog #R0883 (Sigma-Aldrich, Darmstadt, Germany) supplemented with 10% fetal bovine serum Catalog #FB-1001/500 (BioSera, Nuaille, France) and 1% L-glutamine Catalog #G7513-100ML (Sigma-Aldrich, Darmstadt, Germany). All cell lines were maintained at 37° C. in a humidified 5% CO₂ atmosphere. AlamarBlue assay was performed to assess cell viability. The viable cells were seeded at a density of 2×10⁴ (200 μl/well) in 96-well plate and incubated at 37° C. and 5% CO₂ for 24 h to form a cell monolayer. After 24 h of incubation, supernatant on the monolayer was aspirated and 200 μl of medium and varying concentrations of the substances were added and incubated for 24 h, 48 h and 72 h time points. After the specific time points, 20 μl of 0.15 mg/ml resazurin sodium salt Catalog #199303 (Sigma-Aldrich, Darmstadt, Germany) in PBS was added to each well and incubated for 3 h at 37° C. and 5% CO₂. Fluorescence was measured at 560 nm excitation and 590 nm emission wavelength. The experiment was performed in triplicates. The average fluorescence was calculated for each triplicate. One sample t-test was used to determine differences in the mean by comparing the treated samples with the untreated controls. P values <0.05 were considered to indicate a statistically significant difference. Results were calculated using the Microsoft Excel 2016. The measurements from AlamarBlue assay were used to determine the LC50 (median lethal concentration) values by using the Microsoft Excel 2016.

### Results

### Liposomes Preparation and Encapsulation of Nucleoside Analogues

2-(6-Amino-9H-purin-9-yl)quinazolin-4-ol and {[2-(6-amino-9H-purin-9-yl)quinazolin-4-yl]oxy}phosphonic acid were tested at different liposomal systems. At Table 3, the results of the encapsulation efficacy, as calculated according equation (1), of each system can be seen.

| {[2-(6-amino-9H-purin-9-yl)quinazolin-4- | | 2-(6-Amino-9H-purin-9-yl) | |
|---|---|---|---|
| yl]oxy}phosphonic acid | | quinazolin-4-ol | |
| | Encapsulation Efficacy | | Encapsulation Efficacy |
| Code | (EE %) | Code | (EE %) |
| 1 | 60.8 | 1 | 55.2 |
| 2 | 76.8 | 2 | 55.2 |
| 3 | 50.0 | 3 | 65.1 |
| 4 | 60.8 | 4 | 54.4 |
| 5 | 70.4 | 5 | 65.6 |

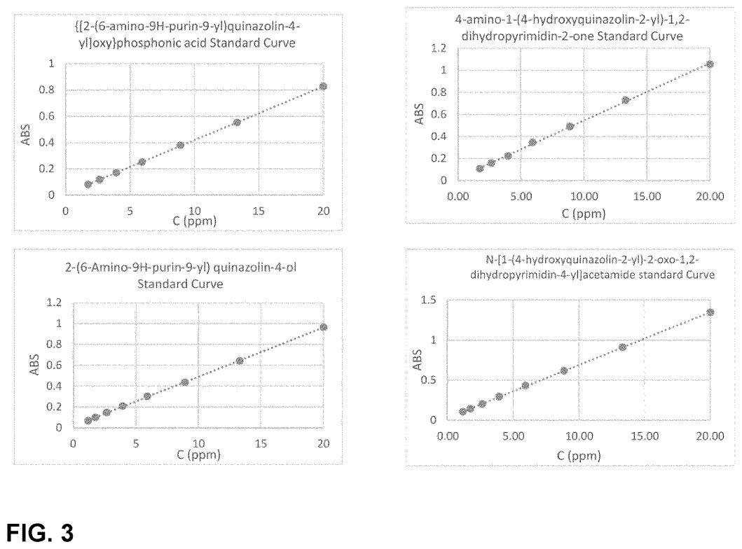

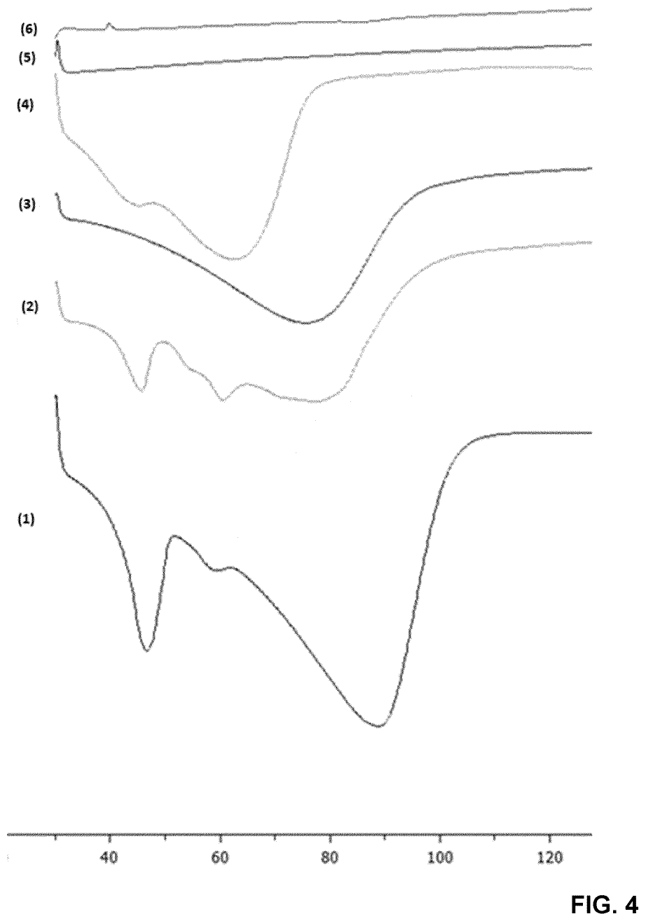

Liposomal systems 2 and 3 exhibited the most successful results for {[2-(6-amino-9H-purin-9-yl)quinazolin-4-yl]oxy}phosphonic acid and 2-(6-Amino-9H-purin-9-yl)quinazolin-4-ol, respectively. These two systems (Liposomal systems 2 and 3) were tested for the rest of nucleoside analogues, and the encapsulation efficacy was calculated indirectly based on equation (1) and the standard curves (FIG. 3).

For example, for {[2-(6-amino-9H-purin-9-yl)quinazolin-4-yl]oxy}phosphonic acid system code 1, after the centrifugation, we keep the supernatant from which, a fraction is collected to prepare a solution with targeted concertation of 10 ppm. Based on the Dilution Equation, C1V1=C2V2, C1=1000, because we consider, at the initial solution that the concentration is 1000 ppm (based on the fact that all of the added nucleoside analogue would be in the supernatant)
C2: is the targeted concentration, here 10 ppm
V2: volume of the final solution, here 10 mL

Thus, we take 100 uL from the supernatant and we dilute it to 10 mL final volume. The sample is quantified by UV/VIS spectrometry and we calculate the actual concentration of the sample. The concentration is converted to mass and we can estimate the final mass of the nucleoside analogue in the supernatant and in the liposome.

Liposomal systems 2 and 3 were used to develop liposomes with the rest of the nucleoside analogues. The standard curves of the nucleoside analogues in PBS can be seen in FIG. 3.

Encapsulation efficacy of nucleoside analogues
encapsulated into liposomal systems 2 and 3

| N-[1-(4-hydroxyquinazolin-2-yl)-2- | | | |
|---|---|---|---|
| oxo-1,2-dihydropyrimidin-4- | | 4-amino-1-(4-hydroxyquinazolin-2- | |
| yl]acetamide | | yl)-1,2-dihydropyrimidin-2-one | |
| | Encapsulation | | Encapsulation |
| Liposomal | Efficacy | Liposomal | Efficacy |
| system | (EE %) | system | (EE %) |
| 2 | 58.1 | 2 | 73.6 |
| 3 | 89.9 | 3 | 76.2 |

As it is seen from the results summarized in the Table above, the liposomal System 3 was found to display a good encapsulation efficiency and was used as the liposomal system for all nucleoside analogues, except for {[2-(6-amino-9H-purin-9-yl)quinazolin-4-yl]oxy}phosphonic acid, for which analogue System 2 was retained.

### Yields

The yield of different liposomal systems was determined based on the mass of the product after the freeze drying cycle. The initial mass of each product is calculated by the lipids and the molecule added.

| Nucleoside Analogue Liposome | Yield (%) |
|---|---|
| N-[1-(4-hydroxyquinazolin-2-yl)-2-oxo-1,2-dihydropyrimidin-4-yl]acetamide * | 87.7 |
| 4-amino-1-(4-hydroxyquinazolin-2-yl)-1,2-dihydropyrimidin-2-one * | 89.3 |
| {[2-(6-amino-9H-purin-9-yl)quinazolin-4-yl]oxy}phosphonic acid ⁺ | 48.4 |
| 2-(6-Amino-9H-purin-9-yl) quinazolin-4-ol * | 77.84 |

* in System 3/
⁺ in System 2

### DSC

Blank liposomes and nucleoside-analogues encapsulated liposomes exhibit similar endo peaks at the beginning of the thermograph. All liposomes, whether loaded or blank, apart from the {[2-(6-amino-9H-purin-9-yl)quinazolin-4-yl]oxy}phosphonic acid liposome of thermograph (7), seem to have the same glass transition temperature, around 35° C.

There are characteristic endothermic peaks observed at liposomes in the range of 40° C. to 100° C., in all cases, apart from the {[2-(6-amino-9H-purin-9-yl)quinazolin-4-yl]oxy}phosphonic acid liposomes. These peaks arise from phosphatidylcholine in these products which is not used in the case of {[2-(6-amino-9H-purin-9-yl)quinazolin-4-yl]oxy}phosphonic acid liposomes. That is why in this liposome, there is a different broad endothermic peak at the beginning of the thermograph. Furthermore, in the DSC thermographs, where the phase transition peaks are visible, it is apparent that since almost all of the liposomes had the same formulation, their peaks display significant changes. It is believed that these changes are attributable to the different chemical nature of each nucleoside analogue.

### SEM

Photographs of the freeze dried liposomes were taken and studied for their morphological properties as well as for their size. All of the liposomes seem to have the same appearance, almost spherical and uniform in size particles. Their size was approximately calculated with the use of the sizing tool of this instrument.

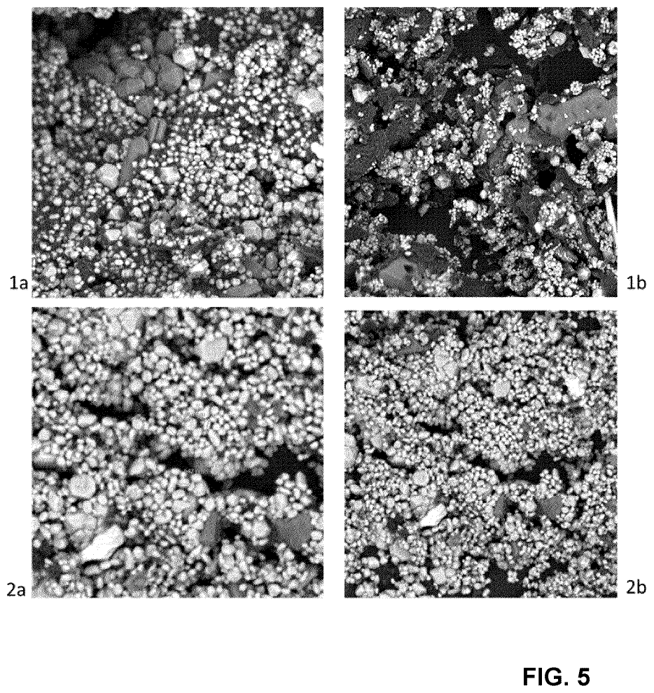

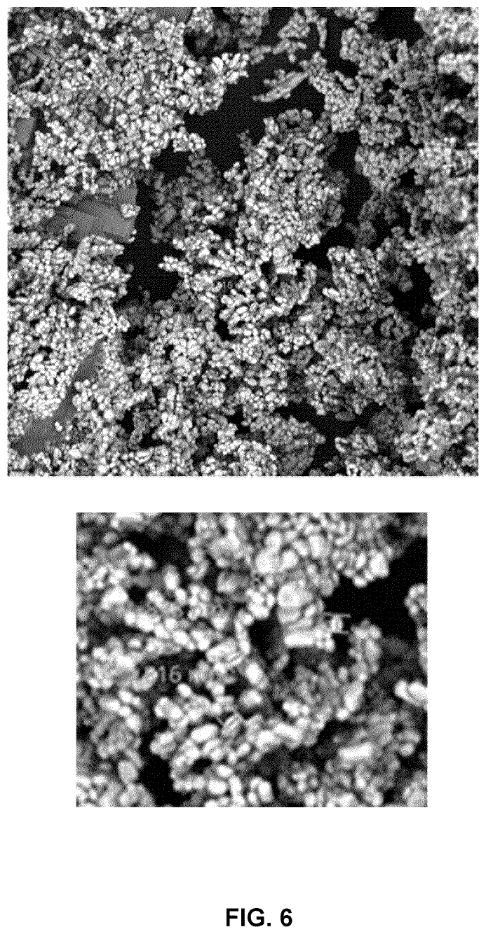

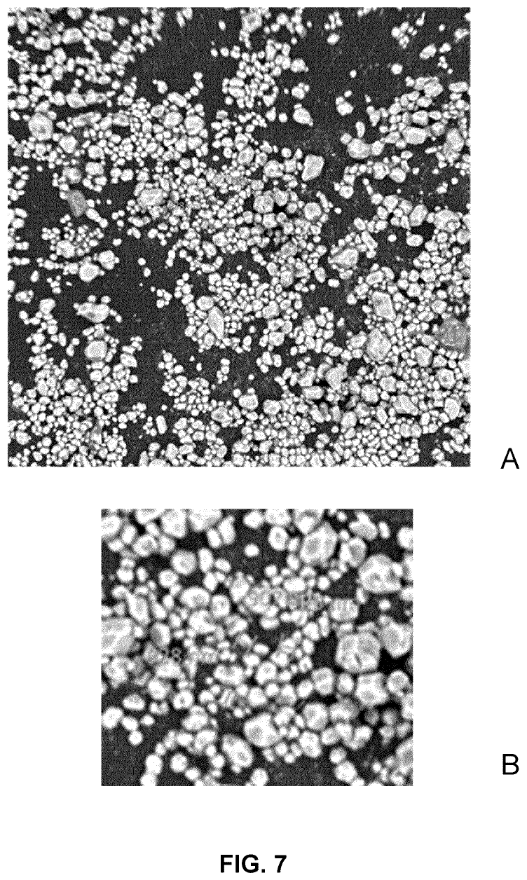

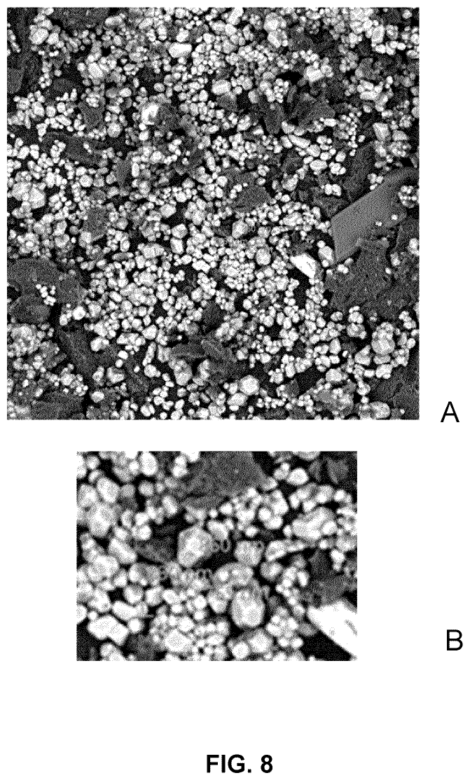

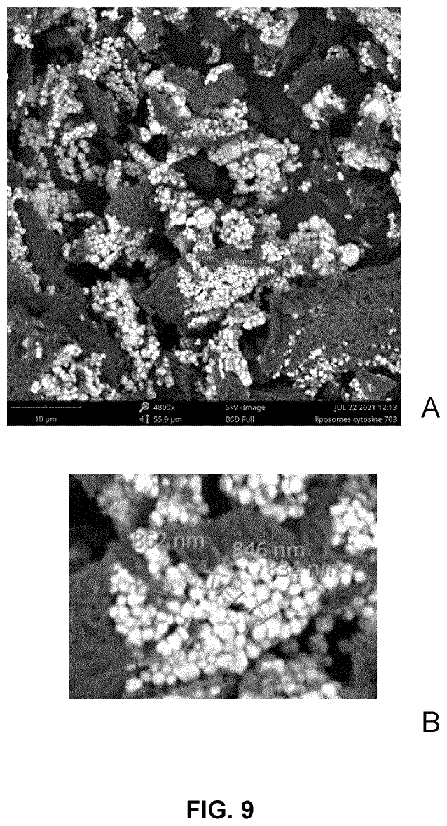

The SEM photographs in FIGS. 5, 6 and 7 show liposomes. The below Table reports liposome size as determined using the sizing tool on the SEM photographs.

| Product | Average Size (nm) | ±SD |
|---|---|---|
| Blank Liposome 1 (System 3) | 713 | 2 |
| Blank Liposome 2 (System 2) | 646.7 | 19.5 |
| Liposome {[2-(6-amino-9H-purin-9-yl)quinazolin-4- | 820.3 | 15.95 |
| yl]oxy}phosphonic acid ⁺ | | |
| Liposome 2-(6-Amino-9H-purin-9-yl) quinazolin-4-ol * | 923.7 | 15.63 |
| Liposome N-[1-(4-hydroxyquinazolin-2-yl)-2-oxo-1,2- | 815.0 | 54.08 |
| dihydropyrimidin-4-yl]acetamide * | | |
| Liposome 4-amino-1-(4-hydroxyquinazolin-2-yl)-1,2- | 847.3 | 14.05 |
| dihydropyrimidin-2-one * | | |

* in System 3/
⁺ in System 2

Based on the above measurements, it can be demonstrated that liposomes encapsulating nucleoside analogues are significantly greater in size, showing that the encapsulation of an active substance inside a liposome can increase its size.

### In Vitro Assessment of Cytotoxic Activity

The below Table illustrates the cytotoxic effects of the five liposomes encapsulating nucleoside analogues against four human cancer cell lines. LC50 values were calculated for the conditions were at least 50% of cell viability was observed in the range of the tested concentrations, 0.01 μM to 10 μM. 2-(6-Amino-9H-purin-9-yl)quinazolin-4-ol, when encapsulated in liposomes, or without being encapsulated in liposomes (not shown in Table) had no statistically significant cytotoxic effect against any human cancer cell lines and at any time points in the range of 0.01 μM to 10 μM. MDA-MB-231 human breast adenocarcinoma cell line exhibited resistance against all of the tested nucleoside analogues, when encapsulated in liposomes or without being encapsulated in liposomes (not shown in Table). For the remaining cancer cell lines, COLO699N, HCT116 and MCF7, no nucleoside analogue had a statistically significant cytotoxic effect when it was used alone (not shown in Table), i.e. without being encapsulated in liposomes. However, when encapsulated in liposomes, for each cancer cell line, specific nucleoside analogues, when encapsulated in liposomes, exhibited significant inhibition of cell growth and in many cases the LC50 was lower than the minimum testing concentration (0.01 μM). In particular, liposomes of 4-amino-1-(4-hydroxyquinazolin-2-yl)-1,2-dihydropyrimidin-2-one and of {[2-(6-amino-9H-purin-9-yl)quinazolin-4-yl]oxy}phosphonic acid in System 3 and 2, respectively, were highly effective in COLO699N, HCT116 and MCF7 at sub-micromole concentrations, as were liposomes of N-[1-(4-hydroxyquinazolin-2-yl)-2-oxo-1,2-dihydropyrimidin-4-yl]acetamide in System 3.

TABLE
LC50 values for the five liposomes encapsulating nucleoside
analogues against four human cancer cell lines.

| | Liposome N-[1- | Liposome | Liposome{[2- | Liposome 2- |
|---|---|---|---|---|
| | (4-hydroxyquinazol | 4-amino-1-(4- | (6-amino-9H- | (6-Amino- |
| | in-2-yl)-2-oxo- | hydroxyquinaz | purin-9- | 9H-purin-9- |
| | 1,2- | olin-2-yl)-1,2- | yl)quinazolin- | yl) |
| | dihydropyrimidin- | dihydropyrimidin- | 4-yl]oxy} phosphonic | quinazolin- |
| LC50 | 4-yl]acetamide | 2-one | acid | 4-ol |
| COLO699N | >10 μM | 0.06 μM (48 h) | 2.03 μM (48 h) | >10 μM |
| HCT116 | 4.06 μM (48 h) | <0.01 μM (48 h) | <0.01 μM (48 h) | >10 μM |
| | | <0.01 μM (72 h) | <0.01 μM (72 h) | |
| MCF7 | >10 μM | <0.01 μM (24 h) | 5.75 μM (24 h) | >10 μM |
| | | 3.56 μM (72 h) | | |
| MDA- | >10 μM | >10 μM | >10 μM | >10 μM |
| MB-231 | | | | |

1. A pharmaceutical composition comprising a nucleoside analogue loaded into liposomes of a liposomal delivery system, wherein the nucleoside analogue is a pyrimidine analogue chosen from 4-amino-1-(4-hydroxyquinazolin-2-yl)-1,2-dihydropyrimidin-2-one or N-[1-(4-hydroxyquinazolin-2-yl)-2-oxo-1,2-dihydropyrimidin-4-yl]acetamide; or a purine analogue chosen from {[2-(6-amino-9H-purin-9-yl)quinazolin-4-yl]oxy}phosphonic acid or 2-(6-amino-9H-purin-9-yl)quinazolin-4-ol.

2. The pharmaceutical composition according to claim 1, wherein the nucleoside analogue is covalently coupled or is not covalently coupled to the liposome.

3. The pharmaceutical composition according to claim 1, wherein the liposomes of the liposomal delivery system have a diameter of at least 750, preferably of at least 775, and more preferably of at least 800 nm, as determined by SEM imaging.

4. The pharmaceutical composition according to claim 1, wherein the liposomes of the liposomal delivery system are sterically stabilized liposomes.

5. The pharmaceutical composition according to claim 1, wherein the liposomes of the liposomal delivery system comprise at least a phospholipid, preferably at least a phosphatidylcholine and/or a phosphoethanolamine, or comprise at least a phospholipid, more preferably at least a phosphatidylcholine and/or a phosphoethanolamine, most preferably in addition to one or more phosphoglycerides and/or one or more sphingolipids.

6. The pharmaceutical composition according to claim 1, wherein the liposomes of the liposomal delivery system comprise, or essentially consist of, as the lipid component(s) of the liposomes, a pegylated phospholipid such as pegylated 1,2-distearoyl-sn-glycero-3-phosphoethanolamine, or a combination of a pegylated and an unpegylated phospholipid such as such as pegylated 1,2-distearoyl-sn-glycero-3-phosphoethanolamine and unpegylated phosphatidylcholine, respectively, optionally together with a sterol such as cholesterol.

7. The pharmaceutical composition according to claim 6, wherein the pegylated phospholipid is pegylated phosphoethanolamine and the unpegylated phospholipid is a phosphatidylcholine.

8. The pharmaceutical composition according to claim 1, wherein the liposomes of the liposomal delivery system are free of fatty acids, in particular free of unsaturated fatty acids, and preferably of monounsaturated fatty acids such as oleic acid.

9. The pharmaceutical composition according to claim 1, wherein the liposomes of the liposomal delivery system comprise an amount of sterol such as cholesterol of from about more than 5 wt % to less than 50 wt %, preferably of from about more than 5 wt % to less than 25 wt %, more preferably of from about more than 5 wt % to less than 20 wt %, most preferably of from about more than 6 wt % to less than 16 wt %, based on the weight of the pegylated phospholipid or the weight of the combination of a pegylated and an unpegylated phospholipid.

10. The pharmaceutical composition according to claim 1, wherein the liposomes of the liposomal delivery system comprises, or consists of, of from 40 to 45 wt % of a pegylated phospholipid such as N-(carbonyl-methoxypolyethylene glycol-2000)-1,2-distearoyl-sn-glycero-3 phosphoethanolamine, of from 40 to 45 wt % of an unpegylated phospholipid such as phosphatidylcholine, and of a sterol such as cholesterol, based on the total weight of the pegylated phospholipid, unpegylated phospholipid and sterol, and preferably wherein the pegylated phospholipid and unpegylated phospholipid are comprised at a weight ratio of 1:1.

11. A pharmaceutical composition according to claim 1 for use in the treatment of colon cancer or lung cancer or breast, preferably via oral or parenteral route.

12. Use of a pharmaceutical composition according to claim 1 for the inhibition of proliferation of cancer cell lines in vitro, preferably in a concentration of 0.01 μM to 10 μM.

13. The use according to claim 12 wherein the cancer cell line is a lung or colon or breast cancer cell line and the nucleoside analog is 4-amino-1-(4-hydroxyquinazolin-2-yl)-1,2-dihydropyrimidin-2-one or {[2-(6-amino-9H-purin-9-yl)quinazolin-4-yl]oxy}phosphonic acid, and preferably the nucleoside analog is present in a concentration of 0.01 μM to 1 μM.

14. A process for producing a liposomal delivery system comprising a nucleoside analogue loaded into liposomes,
wherein the liposomes of the liposomal delivery system comprise, or consist of, a combination of a pegylated and an unpegylated phospholipid, and preferably of from 40 to 45 wt % of a pegylated phospholipid such as N-(carbonyl-methoxypolyethylene glycol-2000)-1,2-distearoyl-sn-glycero-3 phosphoethanolamine, of from 40 to 45 wt % of an unpegylated phospholipid such as phosphatidylcholine, and of a sterol such as cholesterol, based on the total weight of the pegylated phospholipid, unpegylated phospholipid and sterol, and more preferably wherein the pegylated phospholipid and unpegylated phospholipid are comprised at a weight ratio of 1:1, and
wherein the nucleoside analogue is chosen among 4-amino-1-(4-hydroxyquinazolin-2-yl)-1,2-dihydropyrimidin-2-one, or N-[1-(4-hydroxyquinazolin-2-yl)-2-oxo-1,2-dihydropyrimidin-4-yl]acetamide,
wherein the process comprises the steps of:
a. solubilizing the pegylated phospholipid, unpegylated phospholipid, and sterol, and the nucleoside analogue, in an polar solvent, preferably in a mixture of polar solvents,
b. evaporating the solvent, and
c. adding an aqueous solution or water, preferably an aqueous buffer solution, such as to form a first liposomal suspension, and
d. sonicating the first liposomal suspension to form a second liposomal suspension,
e. annealing the second liposomal suspension to form the liposomal delivery system comprising a nucleoside analogue loaded into liposomes.

15. A process for producing a liposomal delivery system comprising a nucleoside analogue loaded into liposomes,
wherein the liposomes of the liposomal delivery system comprise, or consist of, a combination of a pegylated and an unpegylated phospholipid, and preferably of from 40 to 45 wt % of a pegylated phospholipid such as N-(carbonyl-methoxypolyethylene glycol-2000)-1,2-distearoyl-sn-glycero-3 phosphoethanolamine, of from 40 to 45 wt % of an unpegylated phospholipid such as phosphatidylcholine, and of a sterol such as cholesterol, based on the total weight of the pegylated phospholipid, unpegylated phospholipid and sterol, and more preferably wherein the pegylated phospholipid and unpegylated phospholipid are comprised at a weight ratio of 1:1, and
wherein the nucleoside analogue is chosen among {[2-(6-amino-9H-purin-9-yl)quinazolin-4-yl]oxy}phosphonic acid and 2-(6-Amino-9H-purin-9-yl)quinazolin-4-ol,
wherein the process comprises the steps of:
a. solubilizing the pegylated phospholipid, unpegylated phospholipid, and sterol, in an polar solvent, preferably in a mixture of polar solvents,
b. evaporating the solvent, and
c. adding an aqueous solution of the nucleoside analogue, preferably an aqueous buffer solution of the nucleoside analogue, such as to form a first liposomal suspension, and
d. sonicating the first liposomal suspension to form a second liposomal suspension,
e. annealing the second liposomal suspension to form the liposomal delivery system comprising a nucleoside analogue loaded into liposomes.
